(19) United States
(12) Patent Application Publication
(10) Pub. No.: US 2004/0247571 A1
(43) Pub. Date: Dec. 9, 2004

(54) Neural cells expressing tyrosine hydroxylase

(76) Inventors: Xia Meijer, Jarfalla (SE); Mette Gronborg, Ballerup (DK); Lars Wahlberg, Ballerup (DK)

Correspondence Address: BROWDY AND NEIMARK, P.L.L.C., 624 NINTH STREET, NW, SUITE 300, WASHINGTON, DC, 20001-5303, US

(21) Appl. No.: 10/475,334

(22) PCT Filed: Apr. 23, 2002
(86) PCT No.: PCT/DK02/00262

Related U.S. Application Data
(60) Provisional application No. 60/286,084, filed on Apr. 23, 2001.
(60) Provisional application No. 60/289,933, filed on May 9, 2001.

(51) Int. Cl. A61K 48/00; C12N 5/08

(52) U.S. Cl. 424/93.7; 424/93.21; 435/368

(57) ABSTRACT

The invention provides a means for efficiently generating large numbers of TH expressing neural cells for neurotransplantation into a host to treat neurodegenerative disease, neurological trauma, stroke, or in other neurodegenerative disease, neurological trauma, stroke, or in other diseases of the nervous system involving loss of neural cells, particularly Parkinson's disease.

### FIELD OF THE INVENTION

The present invention relates to methods for producing neural cells that express tyrosine hydroxylase and compositions relating to the same.

### BACKGROUND OF THE INVENTION

CNS disorders include, for example, disease states of the CNS, dysfunction of the CNS and acute injuries to the CNS. Alzheimer's disease, Parkinson's disease, depression, epilepsy, schizophrenia, and brain injury, for example, may all be termed CNS disorders. As may be appreciated, any improvement in the treatment of CNS disorders is highly desirable.

In that respect, developing dopaminergic neurons originating from aborted human embryos have previously been implanted in the brains of patients with Parkinson's disease and have successfully restored function (Bjorklund, Novartis Found Symp 2000; 231: 7-15). A method of treating CNS disorders with implanted neurons is, therefore, a promising approach to the treatment of CNS disorders. However, the logistical and ethical problems associated with preparing sufficient numbers of well-characterized fetal cells for the large number of individuals that need such treatment make this therapeutic approach unrealistic. This limitation of fetal cells might be circumvented, however, by the identification of a specific neural cell line capable of being expanded in vitro for cell banking. Such a cell line should be able to differentiate into cells with a neuronal phenotype similar to the nigral dopaminergic neurons. Furthermore, the cells should be able to survive, maintain their dopaminergic phenotype, and function following transplantation and integration into the striatum. With respect to grafting such cells into a mammal in need of such treatment, such techniques are well known to one of skill in the art (for example, U.S. Pat. Nos. 5,082,670 and 5,762,926, both hereby incorporated by reference).

Continuously dividing multipotent cultures of human neural progenitor cells derived from embryonic forebrain tissue remain viable in vitro for at least 35 passages or more than 350 days (Carpenter, Exp Neurol. 1999 August; 158(2): 265-78.). Under serum-free conditions in the presence of epidermal growth factor (EGF), basic fibroblast growth factor (bFGF) and leukaemia inhibitory factor (LIF), these cultures grow as non-adherent clusters (“neurospheres”). When plated on a substrate in medium without mitogens, the cells in the culture differentiate and have the ability to generate the major phenotypes in the CNS (i.e., neurons, astrocytes and oligodendrocytes). The majority of the neurons formed under these conditions, however, are immunoreactive for gamma-amino butyric acid (GABA). Only rarely are neuronal cells expressing tyrosine hydroxylase (TH) observed (Svendsen, Exp Neurol 1997 November; 148(1): 135-46, Carpenter, 1999). TH expression is important because TH catalyzes the rate-limiting step in the biosynthesis of dopamine. Specifically, TH utilizes tyrosine, molecular oxygen and tetrahydrobiopterin as co-substrates in the formation of 3,4-dihydroxyphenylalanine (DOPA). Aromatic amino acid decarboxylase (AADC) then converts DOPA to dopamine (DA). In noradrenergic cells, dopamine is converted to norepinephrine by the enzyme dopamine-β-hydroxylase (DBH). Thus, cells producing dopamine and norepinephrine are both characterized by the expression of TH (and AADC). In contrast, adrenergic cells specifically express DBH, which is not expressed in dopaminergic cells.

It is therefore of great interest to develop methods which not only allow the differentiation of neural progenitor cells in vitro, but do so in such a way that maximizes the percentage of neuronal cells which express TH. U.S. Pat. No. 5,851,832 (hereby incorporated by reference) describes the in vitro growth and proliferation of multipotent neural stem cells and their progeny. However, as compared with the techniques described herein, the methods described therein do not result in a population of neural cells wherein a significant percentage of the cells are TH expressing neurons. U.S. Pat. No. 5,980,885 (hereby incorporated by reference) describes the growth factor induced proliferation of neural precursor cells in vivo. However, the methods described therein are not directed towards the in vitro proliferation of neurons and, as compared with the techniques described herein, do not result in a population of neural cells wherein a significant percentage of the cells are TH expressing neurons. U.S. Pat. No. 5,981,165 (hereby incorporated by reference) describes the in vitro induction of dopaminergic cells. However, as compared with the techniques described herein, the methods described therein do not result in a population of neural cells wherein a significant percentage of the cells are TH expressing neurons. U.S. Pat. No. 5,968,829 and the related U.S. Pat. No. 6,103,530 (both hereby incorporated by reference) describe the use of Leukemia Inhibitory Factor in order to increase the rate of stem cell proliferation or neuronal differentiation. However, as compared with the techniques described herein, the methods described therein do not result in a population of neural cells wherein a significant percentage of the cells are TH expressing neurons. Similarly, U.S. Pat. Nos. 6,040,180, 6,251,669, and 6,277,820 (all incorporated by reference herein) describe methods and uses for neuronal progenitor cells or CNS stem cells. However, as compared with the techniques described herein, the methods described therein do not result in a population of neural cells wherein a significant percentage of the cells are TH expressing neurons. U.S. Pat. No. 6,312,949 describes cells comprising an exogenous nucleic acid Nurr1 that induces TH enzyme synthesis within a cell. However, the methods disclosed therein are directed to elevated TH expression within an individual cell and are distinguished from the methods described herein.

Thus, a need remains in the art for a solution to the known logistical and ethical problems of efficiently preparing sufficient numbers of well-characterized dopaminergic cells. A possible solution would be the identification of a method for producing a specific neural cell line expandable in vitro for cell banking. Such a cell line should be able to efficiently differentiate into cells with a neuronal phenotype similar to the nigral dopaminergic neurons. Furthermore, the cells should be able to survive, maintain their dopaminergic phenotype and function following transplantation and integration into the striatum.

### SUMMARY OF THE INVENTION

The invention provides a method for the in vitro production of a population of neural cells wherein a significant percentage of those cells express tyrosine hydroxylase (TH). In that respect, the invention provides a method for the in vitro production of neural cells expressing TH. The method comprises expanding neural progenitor cells using growth factors and/or by immortalization, plating the cells on a substrate, introducing a defined culture medium containing one or more growth factors belonging to the FGF family, a molecule which gives rise to an increase in intracellular cyclic AMP (cAMP), and an agent stimulating or capable of activating protein kinase C (PKC). The method provides TH expressing cells in significant numbers, similar to that observed in fetal ventral mesencephalon cultures (5-20%). It also provides cells in which the expression of TH is stable after removal of the induction medium. The invention provides a means for generating large numbers of TH expressing neural cells for neurotransplantation into a host in the treatment of CNS disorders, for example, neurodegenerative disease, neurological trauma, stroke, other neurodegenerative diseases, neurological trauma, stroke, and other diseases of the nervous system involving loss of neural cells, particularly Parkinson's disease. Additionally, the TH expressing cells may be used for drug screening or gene expression analysis as would be apparent to one of skill in the art.

### BRIEF DESCRIPTION OF THE DRAWINGS

FIG. 1 depicts cultures of human neural progenitors established from human fetal forebrain (10wFBr991013) plated on PLL/laminin coated coverslips in N2 medium containing aFGF (100 ng/ml), BDNF (50 ng/ml), forskolin (25 μM), TPA (100 nM), dbcAMP (100 μM), GDNF (20 ng/ml), IGFI (100 ng/ml), IL-1α (200 pg/ml). After 3 days incubation, cells were fixed and immunostained for TH. Representative fields using a 20× objective (upper picture) and a 40× objective (lower picture) are shown.

FIG. 2 depicts cultures of human neural progenitors established from human fetal forebrain (10wFBr991013) plated on PLL/laminin coated coverslips in N2 medium containing aFGF (100 ng/ml), forskolin (25 μM), TPA (100 nM) and dbcAMP (100 μM). After 3 days incubation, cells were fixed and immunostained for TH. A representative field using a 20× objective is shown.

FIG. 3 depicts cultures of human neural progenitors established from human fetal forebrain (10wFBr991013) plated on PLL/laminin coated coverslips in N2 medium containing aFGF (100 ng/ml), BDNF (50 ng/ml), forskolin (25 μM), TPA (100 nM), dbcAMP (100 μM), GDNF (20 ng/ml), IGFI (100 ng/ml), IL-1α (200 pg/ml). After 1,3 and 7 days incubations, cells were fixed and immunostained for TH and the percentage of TH positive cells were quantified

FIG. 4 depicts cultures of human neural progenitors established from human fetal forebrain (10wFBr991013) plated on PLL/laminin coated coverslips in N2 medium containing aFGF (100 ng/ml), BDNF (50 ng/ml), forskolin (25 μM), TPA (100 nM), dbcAMP (100 μM), GDNF (20 ng/ml), IGFI (100 ng/ml), IL-1α (200 pg/ml). After 3 days incubation, the medium was changed to N2 medium without any additions. After 3 additional days, cells were fixed and immunostained for TH. A representative field using a 40× objective is shown.

FIG. 5 depicts three electrophoreses. Panel A depicts electrophoresis of PCR products amplified using specific primers for TH (expected size 342 bp) and cDNA generated by reverse transcription of RNA extracted from human neural progenitor cells incubated in induction medium for 1 (T1), 3 (T3) or 7 (T7) days or in 1% FBS for 7 days (F7). Panel B depicts electrophoresis of PCR products amplified using specific primers for AADC (expected size 331 bp) and cDNA generated by reverse transcription of RNA extracted from human neural progenitor cells incubated in induction medium (TH) or 1% FBS (FBS) for 7 days. cDNA generated from adult human Substantia Nigra mRNA (SN) was included as a positive control in Panels A and B. Panel C depicts electrophoresis of PCR products amplified using specific primers for DBH (expected size 440 bp) and cDNA generated by reverse transcription of RNA extracted from human neural progenitor cells incubated in induction medium (TH) or 1% FBS (FBS) for 7 days. cDNA generated from adult human Adrenal Gland (AG) was included as a positive control in Panel C.

FIG. 6 depicts cultures of human neural progenitors established from human fetal forebrain (10wFBr991013) plated on PLL/laminin coated coverslips in N2 medium containing aFGF (100 ng/ml), BDNF (50 ng/ml), forskolin (25 μM), DA (10 μM), TPA (100 nM), dbcAMP (100 μM), GDNF (20 ng/ml), IGFI (100 ng/ml), IL-1α (200 pg/ml). After 7 days incubation, cells were fixed and immunostained for AADC. A representative field using a 20× objective is shown.

FIG. 7 depicts cultures of HNSC.100 cells (human neural progenitor cells immortalized with v-myc) stained for TH. The HNSC.100 cells were seeded on glass coverslips coated with. PLL and laminin in differentiation medium with the following additions: aFGF (100 ng/ml), BDNF (50 ng/ml), forskolin (25 μM), TPA (100 nM), dbcAMP (100 μM), GDNF (20 ng/ml), IGFI (100 ng/ml), IL-1α (200 pg/ml). After 1 day, cells were fixed and stained for TH as described in Example 1. A representative field using a 40× objective is shown.

### DETAILED DESCRIPTION OF THE INVENTION

Although there are reports in the literature describing agents capable of affecting TH expression (as well as more generally affecting survival and differentiation in both non-catecholamine and catecholamine cells), the prior art does not provide methods or compositions for an efficient induction of TH expression in a growth factor-expanded neural culture under defined conditions.

For example, human CNS stem cells differentiate spontaneously upon removal of growth factors. However, the predominant neuronal phenotype generated is GABAergic (Vescovi, 1999). TH-expressing neurons have been generated in small numbers from embryonic forebrain multipotent stem cells by treatment with basic fibroblast growth factor (bFGF/FGF2) in combination with a glial cell conditioned medium (Daadi, J. Neurosci. 1999 June 1; 19(11): 4484-97). TH induction has been achieved in cultures of primary neurons using acidic fibroblast growth factor (aFGF/FGF1), along with various co-activators such as brain or muscle extracts (lacovitti, Neuroreport 1997 Apr. 14; 8(6): 1471-4). As can be seen, to the extent that these methods were able to produce TH expressing neuronal cells at all, the methods were not efficient.

Iacovitti (1997) was able to induce TH in neurons newly differentiated from NT2 cells derived from a human embryonal carcinoma by treating the cells with FGF1 and coactivators including dopamine (DA), 12-O-etradecanoylphorbol-13-acetate (TPA), 3-isobutyl-1-methylxanthine (IBMX) and forskolin. This was not successful in the undifferentiated precursors (NT2) derived from a teratocarcinoma. Furthermore, this approach did not induce TH when attempted with a number of murine and rodent cell lines, including an EGF-propagated neural stem/progenitor cell line grown as neurospheres. Carpenter (1999) reported generating a few TH-positive cells from human neurospheres with an induction protocol using a combination of IL-1b, IL11 and GDNF over a period of 20 days, however neither quantification nor characterization of these cells has been published. More recently Caldwell et al. (Nat Biotechnol. 2001 May;19(5):475-9) found that multiple factors did not generate TH-expressing cells from human neurospheres. In conclusion, the efficient and stable induction of TH in neural progenitor cells has not been achieved to date using a defined medium.

Methods that are successful for TH induction in primary or differentiated neurons have not transferred to neural progenitor cells. The use of cell conditioned media has met with some success in generating TH-expressing progenitor cells, however, the variable nature and unknown content of these media does not lead to readily reproducible results.

Finally, with respect to the NT2 and other tumorigenic cell lines, TH-expressing cells produced from growth factor expanded neural progenitor cells have advantages over TH expressing cells generated from NT2 cells. Cultures generated from NT2 cells are potentially tumorigenic, in addition they require a long TH induction protocol (6 weeks predifferentiation to neurons followed by 5 days exposure to the TH induction cocktail). The TH-expressing cells of the present invention are distinguished from NT2 cells by being normal, non-tumorigenic cells expanded either by growth factors or by well-defined genetic modification. In contrast, NT2 cells are spontaneously immortal, being derived from a metastatic tumor.

The present invention provides methods for producing a population of neural cells in vitro, wherein a proportion or percentage of the cells express tyrosine hydroxylase. In so doing, the invention provides methods that allow the generation of a significant number of TH immunoreactive cells displaying neuronal properties from neural progenitor cultures. According to the methods of the present invention, in vitro production of neural cells expressing tyrosine hydroxylase is achieved by expanding neural progenitor cells using growth factors and/or by immortalization, plating the cells on a substrate, and introducing a defined culture medium to which has been added: one or more growth factors belonging to the FGF family; a molecule which gives or results in an increase in intracellular cAMP; and an agent stimulating or activating PKC.

Neural progenitor cells may be obtained from the adult and developing mammalian CNS, preferably from embryonic brain tissue. They may also be generated from embryonic stem cells. Such techniques as may be required for obtaining neural progenitor cells or for generating neural progenitor cells from stem cells are well known to one of skill in the art. Cultures of neural progenitor cells may be maintained and expanded in the presence of one or more growth factors such as epidermal growth factor (EGF), leukaemia inhibitory factor (LIF) and FGF2 (Carpenter, 1999) or ciliary neurotrophic factor (CNTF). These cells are self-renewing, the cells proliferate for long periods in mitogen containing serum free media, and the cells, when differentiated, comprise a cell population of neurons, astrocytes and oligodendrocytes.

Neurosphere cultures generated from human embryonic forebrain have a significant expansion potential. When grown in the presence of EGF, bFGF and LIF, cell cultures preserve their multipotency, remain viable, and are capable of expansion for more than 30 passages (i.e., at least one year). This can result in a 10⁷ fold increase in cell numbers. Theoretically, such cultures generated from one or only a few fetuses should be sufficient as supply for transplantation of all patients with Parkinson's disease.

Neural progenitor cells immortalized by genetic modification may be grown as adherent cultures or in suspension cultures as “neurospheres”. They may be generated by introduction of an oncogene such as vmyc, or by introduction of DNA sequences expressing a telomerase.

The neural progenitor cells described herein may be immortalized or conditionally immortalized using known techniques. Among the conditional immortalization techniques contemplated are Tet-conditional immortalization (see WO 96/31242, incorporated herein by reference), and Mx-1 conditional immortalization (see WO 96/02646, incorporated herein by reference). A number of immortalized cell lines with the characteristics of neural stem/progenitor cells are described in the literature. Examples include HNSC.100 (Villa et al., 2000; Exp. Neurol. 161; 67-84), H6 cells (Flax et al., 1998; Nat. Biotech. 16, 1033-1039) and RN33B cells (Whittemore and White, 1993; Brain Res. 615, 27-40).

In addition to human embryonic forebrain, neurosphere cultures can also be generated from other regions of the developing brain including the mesencephalon and spinal cord. Although data using rodent tissue indicate that some positional identity (reflected by expression of regional markers) is preserved in the primary neurospheres, subculturing seems to lead to the generation/selection of a more uniform type of neurosphere and loss of regional specificity (Santa-Olalla et al., 2000 Soc. Neurosci. abstract 23.3). Accordingly, the conditions for TH induction of this invention can be applied to neurosphere cultures generated from sources other than human embryonic forebrain. Such cultures could include those generated from the adult human or rodent CNS, or from embryonic stem cells. The cultures produced by the methods of the present invention may be trypsinized and reseeded without losing the TH expressing cells. This makes such cultures a potentially attractive alternative to the fetal transplants used for implantation in Parkinson's patients.

The term “neural stem cell” as used herein refers to an undifferentiated neural cell that can be induced to proliferate using the methods of the present invention. The neural stem cell is capable of self maintenance, meaning that, with each cell division, one daughter cell will also be a stem cell.

The term “neural cell” as used herein refers to neurons, including dopaminergic neurons as well as glial cells, including astrocytes, oligodendrocytes, and microglia.

The term “expanding” is used interchangeably with “proliferating” and as used herein it means cultivation of cells.

The term “progenitor cell” as used herein refers to any cell that can give rise to a distinct cell lineage through cell division. For example, a neural progenitor cell is a parent cell that can give rise to a daughter cell having characteristics similar to a neural cell. A neural progenitor cell may be the non-stem cell progeny of a neural stem cell.

The term “population” as used herein in the context of cells, refers to more than one cell, preferably, many cells. In a preferred usage, a population of cells results from the expansion of similar, or preferably identical cells.

The term “significant percentage”, when used herein to describe the percentage of cells expressing TH in a population of neural cells, refers to a percentage that is higher than that percentage resulting from the methods of the prior art.

The term “substantial percentage”, when used herein to describe the percentage of cells expressing TH in a population of neural cells, refers to a percentage that is higher than that percentage resulting from the methods of the prior art described herein.

The term “improved percentage”, when used herein to describe the percentage of cells expressing TH in a population of neural cells, refers to a percentage which exceeds that percentage of cells expressing TH in a population of neural cells resulting from the spontaneous differentiation of CNS cells upon removal of growth factors.

The term “base line percentage” when used herein, describes the percentage of cells expressing TH in a population of neural cells resulting from the spontaneous differentiation of CNS cells upon removal of growth factors. The term is preferably used with a numerical modifier, for example, “twice the base line percentage” or, in general, any multiplier that exceeds one (i.e., 1.1, 1.5, 2.0 etc.).

The term “catecholamine-related deficiency” as used herein is any physical or mental condition that is associated with or attributed to an abnormal level of a catecholamine such as dopamine. This abnormal level may be restricted to a particular region of the mammal's brain (i.e. midbrain) or adrenal gland. A catecholamine deficiency can be associated with disease states such as Parkinson's disease, manic depression, and schizophrenia. In addition, catecholamine-related deficiencies can be identified using clinical diagnostic procedures.

The term “tyrosine hydroxylase-related deficiency” as used herein is any physical or mental condition that either is associated with underproduction or abnormal production of tyrosine hydroxylase or could be managed or treated by tyrosine hydroxylase expression. TH deficiencies may be associated with disease states such as, for example, Parkinson's disease.

One embodiment of this invention is directed towards a method for producing a population of neural cells in vitro wherein a significant percentage of the cells in the population express tyrosine hydroxylase. This method comprises introducing a population of expanded and plated neural progenitor cells to a defined culture medium, wherein the culture medium comprises: (1) one or more growth factors belonging to the Fibroblast Growth Factor (FGF) family; (2) a molecule which results in the activation of cyclic AMP (cAMP) dependent protein kinase (PKA); and (3) an agent which activates Protein Kinase C (PKC).

Another embodiment of this invention is directed towards a method for producing a population of neural cells in vitro wherein a percentage of the cells of the population express tyrosine hydroxylase, the method comprising the following steps:

(a) expanding a population of neural progenitor cells;

(b) plating the population of neural progenitor cells on a substrate; and

(c) introducing the population of neural progenitor cells to a defined culture medium, said culture medium comprising:

(i) one or more growth factors belonging to the FGF family;

(ii) a molecule which gives an increase in intracellular cAMP; and

(iii) an agent that stimulates PKC.

In one embodiment of the method of this invention, a significant percentage of the cells in the population of produced neural cells express tyrosine hydroxylase.

In another embodiment of the method of this invention, a substantial percentage of the cells in the population of produced neural cells express tyrosine hydroxylase.

In another embodiment of the method of this invention, an improved percentage of the cells in the population of produced neural cells express tyrosine hydroxylase.

In another embodiment of the method of this invention, the percentage of the cells in the population of produced neural cells expressing tyrosine hydroxylase is equal to (n times the base line percentage) where n is greater than one and (n times the base line percentage) does not exceed 100. In a preferred embodiment n is between 2 and 5. In another preferred embodiment, n is between 5 and 10. In another preferred embodiment, n is between 10 and 25.

In another preferred embodiment, n is between 25 and 500. In another embodiment, n is greater than 500. The base line percentage is typically in the order of magnitude 0.1% or less.

In another embodiment of the method of this invention, the percentage of the cells in the population of produced neural cells expressing tyrosine hydroxylase is greater than zero when the baseline percentage is zero.

In a preferred embodiment, the neural progenitor cells are expanded by immortalization through genetic modification.

In a preferred embodiment, the growth factor is selected from the group consisting of EGF, bEGF/FGF2, LIF, and CNTF, or a combination thereof.

In a preferred embodiment, the substrate is selected from the group consisting of PLL, PDL, PON, laminin, fibronectin and collagen, or a combination thereof.

In a preferred embodiment, the substrate contains PLL and laminin or PLL and fibronectin.

In a preferred embodiment, the defined culture medium is DMEM-F12 supplemented with N2 or B27.

In a preferred embodiment, the growth factor belonging to the FGF family is selected from the group consisting of aFGF/FGF-1, bFGF/FGF2, FGF4, and FGF8, or combinations thereof, preferably aFGF/FGF-1 and bFGF/FGF2. Preferably, the concentration of the growth factor belonging to the FGF family in the culture medium is from 1 to 500 ng/ml, more preferably from 10 to 200 ng/ml. When more than one compound is used, each compound is used in the before mentioned concentration.

In a preferred embodiment, the molecule that gives an increase in intracellular cAMP is selected from the group consisting of dbcAMP, IBMX, forskolin, 8-BrcAMP, and CPT cAMP, or combinations thereof.

In a preferred embodiment, the molecule that gives an increase in intracellular cAMP is a combination of forskolin and dbcAMP. Preferably, the concentration of the molecule that gives an increase in intracellular camp in the culture medium is from 10 to 1000 μM, more preferably from 10 to 200 μM. When more than one compound is used, each compound is used in the before mentioned concentration.

In a preferred embodiment, the agent stimulating PKC is selected from the group consisting of TPA, DPT, DPP; bryostatin 1 and mezerein, or combinations thereof, preferably TPA. Preferably, the concentration of the agent stimulating PKC in the culture medium is from 50 to 200 μM, more preferably from 75 to 150 μM. When more than one compound is used, each compound is used in the before mentioned concentration.

In a preferred embodiment, the culture medium further comprises a factor which improves the survival or maturation of the TH expressing neurons.

In a preferred embodiment, the survival or maturation factor is selected from the group consisting of: GDNF Family (GDNF; NTN; ART/NBN); Neurotrophins (BDNF; NT4/5; NGF); Insulins (IGF-I, IGF-II, insulin); and Interleukins (IL-1á; IL-1â); or combinations thereof.

In a preferred embodiment, the percentage of tyrosine hydroxylase expressing cells is significantly increased by further addition of Shh.

In a preferred embodiment, the percentage of the produced cell population expressing tyrosine hydroxylase is greater than 1%, more preferably greater than 2%, more preferably greater than 3%, more preferably greater than 4%, more preferably greater than 5%, more preferably greater than 6%, more preferably greater than 7%, more preferably greater than 8%, more preferably greater than 9%, more preferably greater than 10%, more preferably greater than 11%, and most preferably greater than 12%.

In a preferred embodiment, TH expressing neurons are also immunoreactive for AADC.

In a preferred embodiment, the TH expressing neurons do not express DBH.

In a preferred embodiment, the neural progenitor cells are selected from the group consisting of adult human CNS cells; adult rodent CNS cells; human embryonic cells; human fetal cells; human embryonic or fetal forebrain cells; and embryonic stem cells.

In another embodiment, the invention is directed towards compositions produced according to the method described herein.

In a preferred embodiment, the composition is produced through trypsinization and seeding of the TH expressing cells.

In another embodiment, the invention is directed towards a method for treating a mammal with a tyrosine hydroxylase-related deficiency, such as a disease state of the central nervous system, e.g. Parkinson's disease, comprising administering the composition of this invention directly into the CNS of the mammal, e.g. by transplantation.

The present invention further relates to a culture medium comprising

(a) one or more growth factors belonging to the Fibroblast Growth Factor (FGF) family;

(b) a molecule which results in the activation of cyclic AMP (cAMP) dependent protein kinase (PKA); and

(c) an agent that activates Protein Kinase C (PKC).

The present invention further relates to the use of the composition according of the invention for drug screening. The drug may e.g. be screened for a desired effect on TH expressing cells, such as enhancement of cell survival, increase in TH expression, etc. The present invention further relates to the use of the composition according to the invention for gene expression analysis. Such analysis may e.g. have the purpose of investigating the gene expression profile during neural progenitor cell differentiation or the gene expression profile of the differentiated cell.

Furthermore, the present invention relates to the use of the composition according to the invention for producing antibodies against TH expressing cells. Such antibodies may e.g. be used for screening, identification, isolation and/or cell sorting of biological samples for TH expressing cells.

The invention further relates to the use of the composition according to the invention for investigating the biochemistry and molecular mechanisms of neural progenitor cell differentiation, for example for identifying compounds or genes involved in the induction of progenitor cell differentiation.

Also, the invention relates to a composition according to the invention for use as a pharmaceutical for treating a tyrosine hydroxylase-related deficiency.

Finally, the invention relates to the use of a composition according to the invention for the manufacture of a pharmaceutical for treating a disease state of the central nervous system.

Defined Culture Medium

A defined culture medium contains a variety of essential components required for cell viability, including inorganic salts, carbohydrates, hormones, essential amino acids, vitamins, and the like. Preferably, DMEM or F-12 are used as the standard culture medium, most preferably a 50/50 mixture of DMEM and F-12. Both media and a mixture are commercially available (DMEM-Gibco/LifeTechnologies 61965-026; F-12-Gibco/LifeTechnologies 31765-027; DMEM/F12 (1:1)-Gibco/LifeTechnologies 31331-028). A supplement supporting the survival of neural cells in serum-free medium is added to the medium, preferably N2 or B27 supplement. N2 supplement is commercially available (N2-Gibco/LifeTechnologies 17502-048) and contains insulin 5 μg/ml, transferrin 100 μg/ml, progesterone 6.3 ng/ml, putrescine 16.11 μg/ml and selenite 5.2 ng/ml. B27 supplement is commercially available (B27-Gibco/LifeTechnologies 17504-044) and is a proprietary modification of Brewer's B18 formulation (Brewer, 1989; Brain Res. 494:65). Preferably, the conditions for culturing should be as close to physiological as possible. The pH of the culture medium is typically between 6-8, preferably about 7, most preferably about 7.4. Cells are typically cultured between 30-40° C., preferably between 32-38° C., most preferably between 35-37° C. Cells are preferably grown in 5% CO₂.

Substrates

Plating neurosphere cultures on a charged substrate like polyomithine (PON) allows a significant fraction (10-50%) of the cells to become neurons (Carpenter et al., 1999; Ostenfeld et al., Exp Neurol 2000 July; 164(1): 215-26). Signals derived from the extracellular matrix have significant influences on neuron differentiation and development. In the present invention, a mixture of poly-L-Lysine (PLL) and laminin is used as a substrate for the cells, as laminin is known to promote firm attachment and extensive neurite outgrowth in many neuronal cell cultures (Poltorak et al., Exp Neurol 1992 August; 117(2): 176-84; Ernsberger and Rohrer, Dev Biol 1988 April; 126(2): 420-32; Savettieri et al., Cell Mol Neurobiol 1998 August; 18(4): 369-78). Furthermore, cells are seeded as small spheres which are formed in proliferation medium 5-7 days after dissociation to single cells. When the cell suspension is seeded on suitable substrate and mitogens omitted from the medium, within 10 hours β-tubulin positive cells can be observed on top of cells with glial morphology of which some are migrating out from the core of the sphere. Likewise, TH-expressing cells are observed very early after plating for differentiation. In contrast, dissociation of the spheres to single cell suspension only result in no or very few TH positive neurons and only at high cell density. This suggests that preservation of cell-cell contact is important and is consistent with the observation of high numbers of TH immunoreactive cells in patches within the culture. The need for cell-cell interaction may be explained by the possibility that many of the effects of the inducing factors are mediated through the glial cells present in the culture. In addition to PON and PLL, poly-D-lysine (PDL) may be used as a charged substrate. PDL, by virtue of its stereoisomerism cannot be metabolized by the cells.

Laminin is an example of an extracellular matrix protein. Other examples include fibronectin, tenascin, janusin, and collagen. These have been associated with the maintenance and differentiation of neurons in vitro (Lochter, Eur J Neurosci 1994 Apr. 1; 6(4): 597-606) and could also be used in the differentiation of the cells of this invention.

Growth factors belonging to the Fibroblast growth factor (FGF) family have been found to be important in the development of dopaminergic neurons. These include aFGF/FGF-1, bFGF/FGF2, FGF4, FGF8.

Molecules which gives rise to an increase in intracellular cyclic AMP (cAMP) include 3-isobutyl-1-methylxanthine (IBMX), forskolin, and cAMP derivatives; 8-bromo-cAMP (8br-cAMP), 8-(4-chlorophenylthio)-cAMP (CPT-cAMP), N⁶, 2′-O-dibutyryl cAMP (dbt-cAMP). The effects of increasing intracellular cAMP have been attributed to activation of cAMP-dependent protein kinase (PKA) (Frodin, J Biol Chem 1994 Feb. 25; 269(8): 6207-14).

Activators of protein kinase C (PKC) include the phorbol esters; 12-O-tetradecanoylphorbol-13-acetate (TPA), 12-deoxyphorbol-13-tetradecanoate (DPT) 12-deoxyphorbol-13-phenylacetate (DPP); bryostatin 1 and mezerein (Huguet, Eur J Pharmacol 2000 Dec. 20; 410(1): 69-81).

Factors which improve the survival and maturation of the TH expressing neurons may also be added to the culture medium. These factors include members of the Glial cell-line Derived Neurotrophic Factor (GDNF) family; GDNF; Neurturin (NTN); Artemin/Neublastin (ART/NBN); Neurotrophins; Brain Derived Neurotrophic Factor (BDNF); Neurotrophic Factors (NT4/5); Nerve Growth Factor (NGF); Insulins (IGF-I, IGF-II, insulin); Interleukins (IL-1á; IL-1â).

Sonic hedgehog (Shh), a developmental signaling protein believed to be involved in the development and survival of dopaminergic cells. It has recently been reported that the expression of TH in the developing midbrain is mediated by the synergy of FGF8 and Shh (Ye, Cell. 1998 May 29; 93(5): 755-66). More recently attempts use this combination in vitro induced TH expression in fewer than 2% of NT2/hNT cells. (lacovitti, Exp Neurol 2001 May; 169(1): 36-43).

The neural cells of this invention have numerous uses, including for drug screening, diagnostics, genomics and transplantation. The cells of this invention may be transplanted “naked” into patients according to conventional techniques, into the CNS, as described for example, in U.S. Pat. Nos. 5,082,670 and 5,618,531, each incorporated herein by reference, or into any other suitable site in the body. In one embodiment of the present invention, the cultures containing TH-expressing cells are transplanted directly into the CNS. Parenchymal and intrathecal sites are contemplated. It will be appreciated that the exact location in the CNS will vary according to the disease state. The cells may also be encapsulated and used to deliver biologically active molecules, according to known encapsulation technologies (see, e.g., U.S. Pat. Nos. 4,352,883; 4,353,888; and 5,084,350, each incorporated herein by reference).

The following examples are provided for illustrative purposes only, and are not intended to limit the scope of the claims in any way.

### EXAMPLES

### Example 1

### Induction of TH Immunoreactivity in Growth Factor Expanded Human Forebrain Cultures

Cultures of human neural progenitors established from human fetal forebrain expanded in N2 medium supplemented with EGF/bFGF/LIF or EGF/bFGF/CNTF as indicated were mechanically passaged and maintained in expansion medium for 5-7 days. The small spheres were then plated on glass coverslips coated with poly-L-lysine (PLL, 100 μg/ml) and laminin (50 μg/ml) at a cell density of 100.000 cells/cm² in N2 medium containing 1% FBS for “default differentiation” or in N2 medium supplemented with aFGF (100 ng/ml), BDNF (50 ng/ml), forskolin (25 μM), DA (10 μM), TPA (100 nM), dbcAMP (100 μM), GDNF (20 ng/ml), IGFI (100 ng/ml), IL-1α (200 pg/ml). N2 medium consists of DMEM:F12 (1:1) supplemented with N2 (insulin, transferrin, selenium, progesterone and putrescine), 0.6% glucose and 5 mM HEPES. After 3 days incubation, cells were fixed in 4% paraformaldehyde in PBS for 20 min at room temperature. The cells were washed three times with PBS, followed by overnight incubation with primary antibody (rabbit anti-TH, PelFreez 1:100 or Chemicon 1:800) diluted in PBS incubation buffer which contained 10% normal goat serum, 0.3% Triton X-100 (Sigma) and 1% BSA at 4° C. in a humidified chamber. The cells were washed with PBS, and incubated for 1 hr at room temperature in the dark with secondary antibody (anti-rabbit Cy3, (Chemicon 1:500) diluted in incubation buffer. After washing with PBS, nuclei were counterstained with DAPI or Hoechst 33342. Negative controls (omission of the primary antibody) were included in each experiment.

To quantify the percentage of TH cells, cells were counted in at least three fields from three to six independent coverslips randomly chosen using a 20× objective. The number of TH-immunoreactive cells in each field was counted. The total cell number was obtained by counting nuclei counterstained with DAPI or Hoechst 33342.

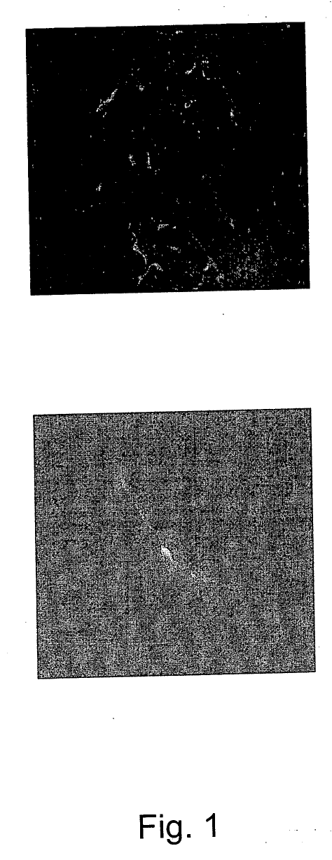

No TH positive cells could be detected in the “default differentiated” cultures. In contrast, approximately 4-10% of the cells became immunoreactive to TH after 3 days. As seen in Table I, passaging of the cultures had no significant effect on the efficiency of TH induction as the number of TH positive neurons generated in a culture after 28 passages was similar as in a culture only passaged for 2 times. Although, a much more efficient expansion of cultures was achieved in medium containing LIF or CNTF, the presence of these growth factors during expansion was dispensable for induction of TH positive cells. In a culture expanded in bFGF/EGF, 8.69±1.12% TH positive cells were observed as compared with a parallel culture established from the same case in bFGF/EGF/LIF 9.85±1.23%. Furthermore, the ability to induce TH expression seems to be a general phenomenon of human neural progenitor/stem cell cultures generated from different regions including cortex and subcortex and is not developmentally dependent as TH induction was observed in cultures generated from tissue of different gestational ages from 6 to 10 weeks (data not shown). Many of the TH positive cells showed the neuronal bipolar morphology seen in FIG. 1 (lower picture).

TABLE 1

| Cell cultures TH-induced 3 days | % TH |
|---|---|
| 10FBr₉₉₀₄₁₉ P2 EGF/bFGF/LIF | 4.7 ± 0.2 |
| 9FBr₀₀₀₁₂₆ P28 EGF/bFGF/LIF | 4.2 ± 0.5 |
| 9FBr₀₀₀₁₂₆ P11 EGF/bFGF/LIF | 4.8 ± 0.1 |
| 10FBr₉₉₁₀₁₃ P24 EGF/bFGF/CNTF | 4.1 ± 0.5 |

### Example 2

### Importance of Substrate

Human neural progenitor cultures expanded in bFGF and CNTF (11.5 wCTX 001115 cells) or in EGF, bFGF and LIF (10wHFBr991013) were seeded after trituration or as small spheres on 12 mm coverslip coated with different substrates at a cell density of 200,000/well in N2 medium containing the TH inductive factors described in Example 1. The different substrate tested was: Poly-L-Ornithine (PLO, 100 μg/ml); PLL (100 μg/ml); PLL combined with lamimin (as above); and PLL (100 μg/ml) combined with fibronectin (50 μg/ml). After incubation for 3 days, cells were fixed in 4% PFA and immunostained for TH as described in Example 1.

TH positive cells were seen on all substrates, although cells (10 wHFBr991013 spheres) plated onto Poly-L-Ornithine (PLO) or PLL alone did not migrate well. No difference of TH induction was seen between the cells plated onto PLL/laminin and PLL/fibronectin. Likewise, quantification indicated that a similar number of TH positive cells were induced on PLL/fibronectin and PLL/laminin from the 11.5CTX001115 cells:

| | |
|---|---|
| PLL/fibronectin | 10.6822% |
| PLL/laminin | 10.6845% |

### Example 3

### Importance of the Various Factors For TH Induction

Human neural progenitor cells expanded in EGF/bFGF/LIF (991013FBr) cells were seeded as small spheres (5 days after trituration to single cell suspension) on PLL/laminin coated 12 mm coverslips at a cell density of 100.000 cells/cm² in N2 medium containing different combinations of the factors described in Example 1. After incubation for 3 days, cells were fixed in 4% PFA and immunostained for TH as described in Example 1.

The results of these experiments indicated that:

1) If Forskolin, dbcAMP or TPA are omitted from the cocktail described in Example 1, significantly fewer TH immunoreactive cells are observed Table 2);

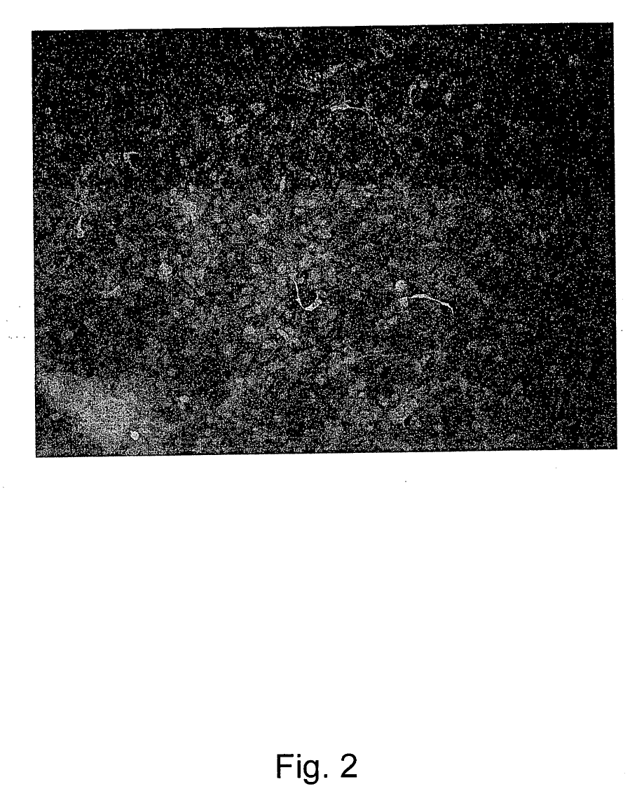

2) Same numbers of TH positive cells with a similar morphology as observed with the cocktail described in Example 1 can be induced with a cocktail consisting of aFGF (100 ng/ml), Forskolin (25 μM), dbcAMP (100 μM) and TPA (100 nM) (Table 2, FIG. 2);

3) Omitting one of the following factors did not affect induction of TH positive cells: BDNF, IGF-I, GDNF, dopamine and IL-1α. FIG. 1 shows a culture induced to express TH in the absence of dopamine; and

4) FGF-1/aFGF maybe replaced with FGF-2/bFGF in the same concentration (100 ng/ml) without any effect on numbers or morphology of TH immunoreactive cells

TABLE 2

| Factors | % TH |
|---|---|
| Standard* | 9.43 ± 0.39 |
| Standard without Forskolin | 5.18 ± 0.46 |
| Standard without dbcAMP | 5.30 ± 0.88 |
| Standard without Forskolin/dbcAMP | 3.86 ± 0.30 |
| Standard without TPA | 0.61 ± 0.21 |
| aFGF, Forskolin, dbcAMP, TPA | 8.69 ± 0.45 |

*aFGF (100 ng/ml), BDNF (50 ng/ml), forskolin (25 μM), TPA (100 nM), dbcAMP (100 μM), GDNF (20 ng/ml). IGFI (100 ng/ml), IL-1α (200 pg/ml)

### Example 4

### Additional Factors may Further Increase the Number of TH Positive Cells

Human neural progenitor cells were seeded as small spheres (5 days after trituration to single cell suspension) on PLL/laminin coated 12 mm coverslips at a cell density of 100.000 cells/cm² in N2 medium containing aFGF (100 ng/ml), forskolin (25 μM), DA (10 μM), TPA (100 nM), dbcAMP (100 μM), GDNF (20 ng/ml), IGFII (100 ng/ml), IL-1α (200 pg/ml) (small cocktail) or aFGF (100 ng/ml), forskolin (25 μM), DA (10 μM), TPA (100 nM), dbcAMP (100 μM), GDNF (20 ng/ml), IGFI (100 ng/ml), IL-1α (200 pg/ml), FGF8b (100 ng/ml) and SHH (100 ng/ml) (big cocktail). After incubation for 3 days, cells were fixed in 4% PFA and immunostained for TH as described in Example 1.

A statistically significant increase (25%) in the number of TH positive neurons was observed after addition of SHH and FGF-8b to the small cocktail:

% TH positive cells in cultures treated with the small cocktail: 9.974±1.198

% TH positive cells in cultures treated with the big cocktail: 12.938±1.198.

### Example 5

### Time Course Of TH Induction

Human neural progenitor cells expanded in EGF/bFGF/LIF (991013FBr) cells were seeded as small spheres (5 days after trituration to single cell suspension) on PLL/laminin coated 12 mm coverslips at a cell density of 100.000 cells/cm² in N2 medium containing the factors described in Example 1 except that dopamine was omitted from the cocktail. Parallel cultures were incubated for 1-7 days. Half of the medium was changed every other day.

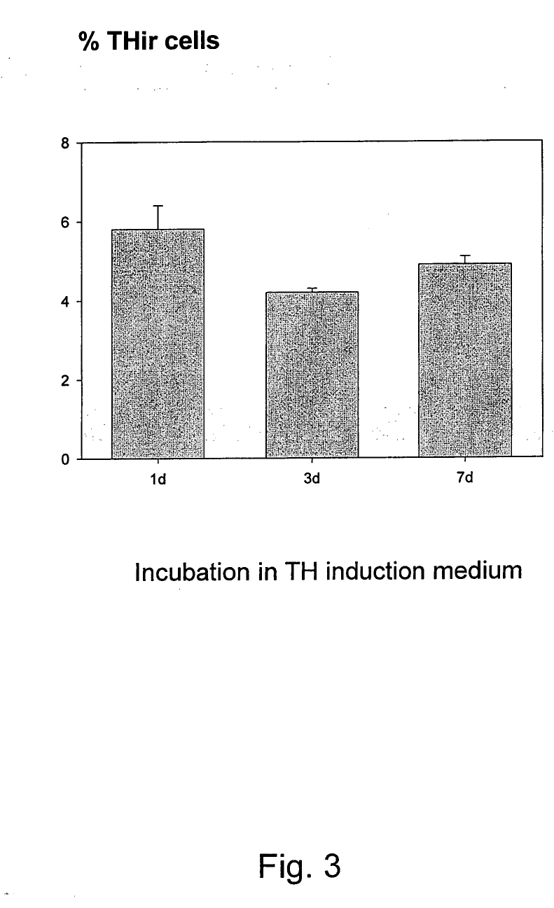

As seen in FIG. 3, maximal TH induction was achieved already after 1 day of exposure to the TH cocktail and sustained for at least 7 days. Although there were no increase in numbers, maturation of the TH positive cells towards a more differentiated neuronal phenotype with longer processes was observed after 3 and 7 days of TH induction.

### Example 6

### Stability of the TH Positive Phenotype

Human neural progenitor cells expanded in EGF/bFGF/LIF (991013FBr) cells were seeded as small spheres (5 days after trituration to single cell suspension) on PLL/laminin coated 12 mm coverslips at a cell density of 100.000 cells/cm² in N2 medium containing the factors described in Example 1 except that dopamine was omitted from the cocktail. After incubation for 3 days, the induction medium was removed and N2 medium supplemented with 1) GDNF, 2) GDNF+IGF-I, 3) 1% FBS+GDNF, 4) 1% FBS, 5) IGF-I, 6) no additions or 7) fresh TH-induction medium were added to parallel TH induced cultures. After additional three days of culturing, cells were fixed and stained for TH as described in Example 1.

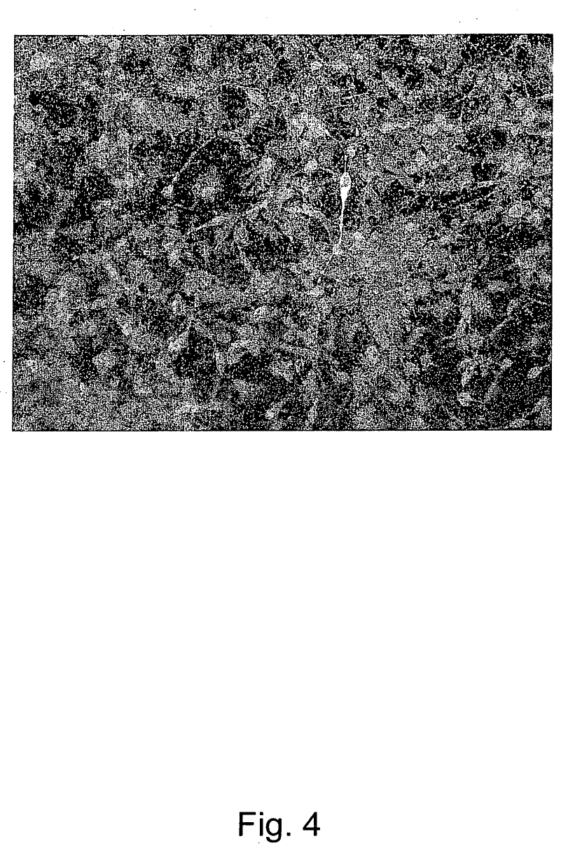

It was possible to find TH-positive cells after changing to all the media described above. TH-positive cells displaying long, elaborate processes were even observed in serum free medium without any additions as seen in FIG. 4.

In another experiment, cells were seeded for TH induction in T₂₅ flasks at a cell density of 100.000 cells/cm². After exposure to induction medium described in Example 1 but without dopamine for 3 days, the induction medium was removed and cells were washed with PBS (without Ca²⁺ and Mg⁺). Then Trypsin-EDTA (0.05% Trypsin, 0.53 mM EDTA Life Technologies 25300-054), and cells were incubated for 5 minutes at 37° C. The flask was tapped manually to loosen the cells from the surface. The trypsin was inactivated by adding N2-medium with 10% FBS and the cells collected by centrifugation for 5 minutes at 1500 rpm. Then the cells were replated onto PLL-lysine/laminin coated coverslips in N2 medium with or without different additives. After additional three days of culturing, cells were fixed and stained for TH. Cells exposed to induction medium without replating for 6 days were included as positive controls.

It was possible to find TH-positive cells in all the seven different media including N2 medium without additions. In one experiment, 3.6% TH-positive cells were observed after replating in induction medium, 3.0% TH-positive cells replated in medium with no additions, and 1.5% TH-positive cells replated in medium with 1% FBS.

Thus, the induction of TH is stable and the TH positive cells survive trypsinization and replating in medium without TH inductive factors.

### Example 7

### Further Characterization of TH-Induced Cells

Cultures of human neural progenitor cells expanded in bFGF/EGF/LIF (10wFBr991013) were seeded in 100-mm dishes coated with PLL/laminin as small spheres (5 days after trituration to single cell suspension) at a cell density of 100,000/cm² in N2 medium containing TH induction factors, as described in Example 1, except that dopamine was omitted from the cocktail. Half of the medium was changed every other day. After 7 days incubation in TH induction medium, total RNA was prepared using Trizol according to the manufacturers protocol (Gibco-BRL). After treatment with DNase, total RNA was reverse transcribed into cDNA with Superscript II RNase H using random hexamer primers (Amersham Pharmacia) according to the manufacturers instructions (Gibco-BRL). The PCR reactions were carried out in a 15-μl volume containing 0.5 unit of Taq polymerase (Amersham Pharmacia), 50 mM Tris-HCl, pH 7.5, 50 mM KCl, 1.5 mM MgCl₂, 10 pmol of specific primer, 200 μM of each of the dNTPs, and RT product equivalent to 125 ng total RNA. The PCR was run for 25-35 cycles and the thermal profile used following a pre-denaturation step at 94° C. for 5 min were specific for the individual primer sets.

Primers:

TH: 5′ GCCCCCACCTGGAGTACTT3′ and 5′GCGTGGCGTATACCTCCTTC3′ (94° C. 30″; 57° C. 30″; 72° C. 30″) resulting in a product of 344 bp

AADC: 5′CGGCATTGGCAGATACCACT 3′ and

5′ ATTCCACCGTGCGAGAACAG 3′ (94° C. 30″; 53° C. 30″; 72° C. 30″) resulting in a product of 331 bp

DBH: 5′CACGTACTGGTGCTACATTAAGGAGC 3′ and

5′ AATGGCCATCACTGGCGTGTACACC 3′ (94° C. 30″; 68° C. 30″; 72° C. 30″) resulting in a product of 440 bp

The PCR products were separated on a 2% agarose gel and visualized by ethidium bromide.

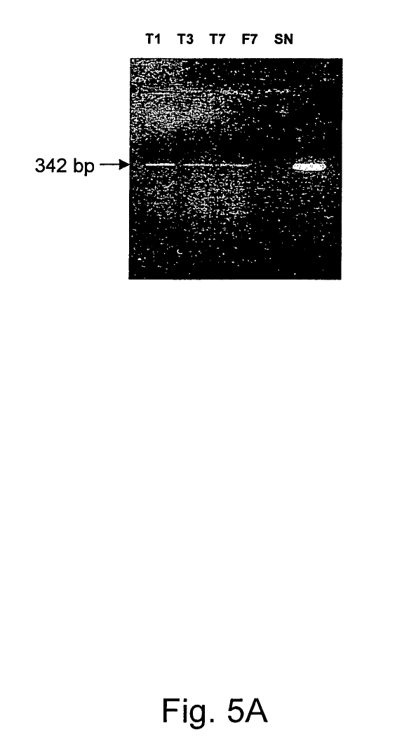

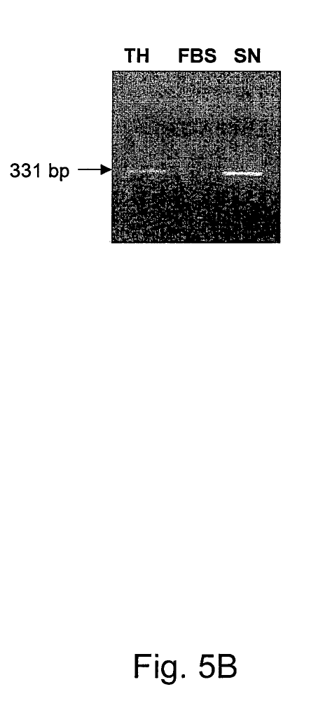

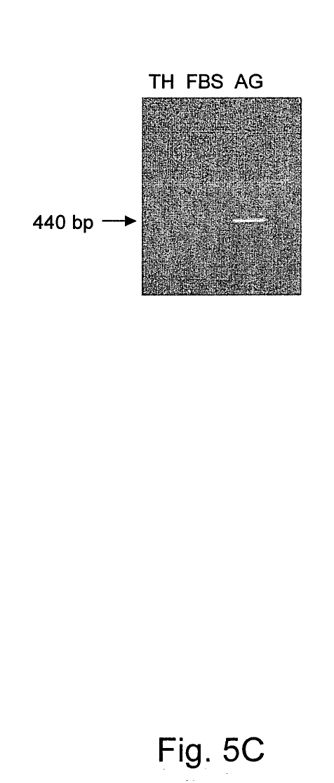

As seen in FIG. 5 (Panels A-C), by using cDNA generated by reverse transcription of RNA extracted from cells that had been incubated for 7 days under conditions inducing TH immunoreactivity, PCR products of the expected sizes could be amplified with primers specific for TH and AADC but not DBH cDNA. These results are consistent with the presence of cells in the TH induced cultures expressing mRNA encoding dopaminergic rather than noradrenergic/adrenergic enzymatic marker proteins.

### Example 8

### Detection of AADC Immunoreactivity In TH-Induced Cultures

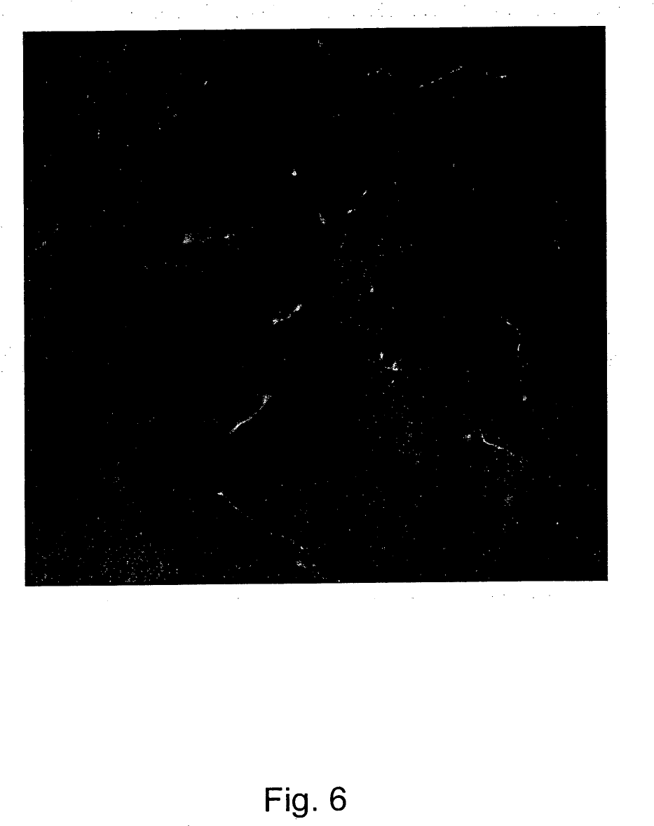

Human neural progenitor cells expanded in EGF/bFGF/LIF (991013FBr) cells were seeded as small spheres (5 days after trituration to single cell suspension) on PLL/laminin coated 12 mm coverslip at a cell density of 100,000/cm² in N2 medium containing the factors described in Example 1, except that dopamine was omitted from the cocktail. After incubation for 7 days, cells were fixed in 4% PFA and immunostained for AADC as described in Example 1, except that a rabbit anti-AADC antibody (Chemicon) diluted 1:2000 was used as primary antibody. As seen in FIG. 6, cells staining brightly for AADC were observed in the TH induced cultures supporting the expression data obtained in Example 7.

### Example 9

### Induction of TH Immunoreactivity in a Human Neural Progenitor Cell Line Immortalized with V-MYC

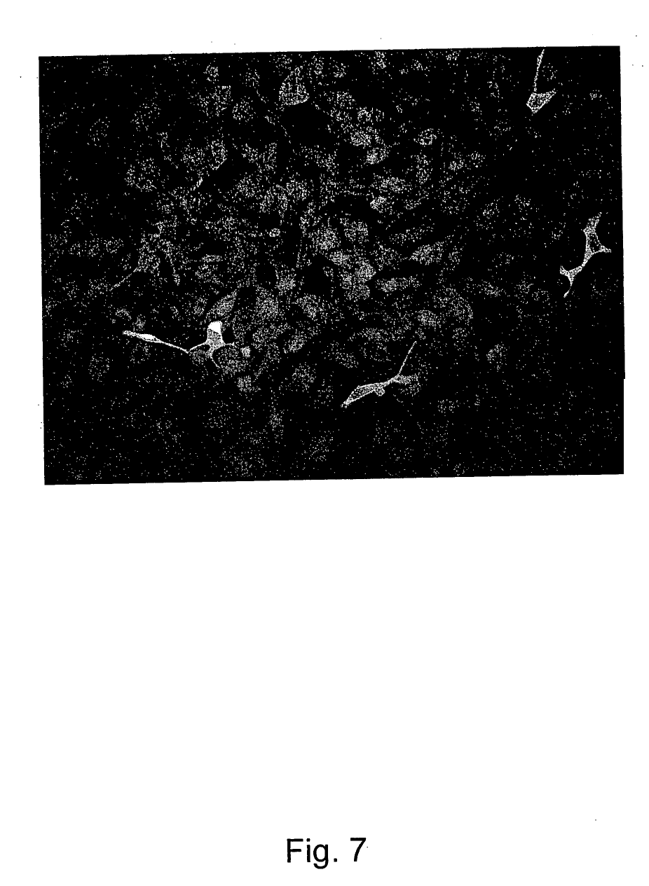

HNSC.100 cells were grown in DMEM:F12 medium supplemented N2, 1% BSA, 20 ng/ml EGF and 20 ng/ml bFGF as adherent cultures in PLL (10 μg/ml) coated TC flasks. For seeding on glass coverslips coated with PLL (50 μg/ml) and laminin (25 μg/ml), cells were trypsinized and plated at a cell density of 25,000 cells/cm² in differentiation medium (growth medium without EGF and bFGF) with or without the following additions: aFGF (100 ng/ml), BDNF (50 ng/ml), forskolin (25 μM), TPA (100 nM), dbcAMP (100 μM), GDNF (20 ng/ml), IGFI (100 ng/ml), IL-1α (200 pg/ml). After 1 day, cells were fixed and stained for TH as described in Example 1. The result of this experiment shown in FIG. 7 demonstrate that also a human neural progenitor cell line immortalized with v-myc can be induced to generate cells staining brightly for TH immunoreactivity, when exposed to the combination of growth factors and signaling molecules described in the example. In contrast, a control culture incubated without the factors added, no TH positive neurons could be found (data not shown).

Equivalents

From the foregoing detailed description of the specific embodiments of the invention, it should be apparent that unique methods and compositions have been described. Although particular embodiments have been disclosed herein in detail, this has been done by way of example for purposes of illustration only, and is not intended to be limiting with respect to the scope of the appended claims that follow. In particular, it is contemplated by the inventor that various substitutions, alterations, and modifications may be made to the invention without departing from the spirit and scope of the invention as defined by the claims. For instance, the choice of the particular cell, substrate, or the particular factors used is believed to be a matter of routine for a person of ordinary skill in the art with knowledge of the embodiments described herein.

1-42. (canceled)

43. A method for producing a population of neural cells in vitro wherein a percentage of the cells of the population express tyrosine hydroxylase (TH), the method comprising the following steps:
(a) expanding a population of neural progenitor cells for 5-7 days after dissociation to single cells to form small spheres;
(b) plating the population of neural progenitor cells on a substrate as small spheres; and
(c) introducing the population of neural progenitor cells to a defined culture medium, said culture medium comprising:
(i) one or more growth factors belonging to the fibroblast growth factor (FGF) family;
(ii) a molecule which gives an increase in intracellular cyclic AMP (camp); and
(iii) an agent that stimulates protein kinase C (PKC).

44. The method of claim 43, wherein a significant percentage of the cells in the population express tyrosine hydroxylase.

45. The method of claim 43, wherein the neural progenitor cells are expanded using at least one growth factor.

46. The method of claim 43, wherein the neural progenitor cells are expanded by immortalization through genetic modification.

47. The method of claim 45, wherein the growth factor is selected from the group consisting of epidermal growth factor (EGF), basic fibroblast growth factor (bEGF), fibroblast growth factor −2 (FGF2), Leukemia Inhibitory Factor (LIF), and Ciliary Neurotropic Factor (CNTF), or a combination thereof.

48. The method of claim 43, wherein the substrate is selected from the group consisting of Poly-L-Lysine (PLL), Poly-D-Lysine (PDL), poly-ornithine (PON), laminin, fibronectin and collagen, or a combination thereof.

49. The method of claim 48, wherein the substrate contains PLL and laminin or PLL and fibronectin.

50. The method of claim 43, wherein the defined culture medium is Dulbecco modified eagles medium F-12 (DMEM-F12) supplemented with N2 or B27.

51. The method of claim 43, wherein the growth factor belonging to the FGF family is selected from the group consisting of aFGF/FGF-1, bFGF/FGF2, FGF4, and FGF8, or combinations thereof.

52. The method of claim 43, wherein the molecule which gives an increase in intracellular cAMP is selected from the group consisting of 2′-o-dibutyryl camp (dbtcAMP), 3-isobutyl-1-methylxanithine, (IBMX), forskolin, 8-bromo-cAMP (8-BrcAMP), and 8-(4-chlorphenylthio(-cAMP)(CPT camp), or combinations thereof.

53. The method of claim 52, wherein the molecule that gives an increase in intracellular cAMP is a combination of forskolin and dbtcAMP.

54. The method of claim 43, wherein the agent stimulating PKC is selected from the group consisting of 12-O-tetradecanoylphorbol-13-acetate (TPA), 12-dioxyphorbol-13-tetradecanoate ((DPT), 12-dioxyphorbol-13-phenylacetate (DPP); bryostatin 1 and mezerein, or combinations thereof.

55. The method of claim 43, wherein the culture medium further comprises a factor that improves the survival or maturation of the TH expressing neurons.

56. The method of claim 55, wherein the survival or maturation factor is selected from the group consisting of glial cell-line derived neurotrophic factor (GDNF), Neurotrophins, Insulins and Interleukins or combinations thereof.

57. The method of claim 43, wherein the percentage of tyrosine hydroxylase expressing cells is significantly increased by further addition of sonic hedgehog (Shh).

58. The method of claim 43, wherein the percentage of the cells in the produced cell population expressing tyrosine hydroxylase is greater than 1%.

59. The method of claim 43, wherein the percentage of the cells in the produced cell population expressing tyrosine hydroxylase is greater than 5%.

60. The method of claim 43, wherein the percentage of the cells in the produced cell population expressing tyrosine hydroxylase is greater than 10%.

61. The method of claim 43, wherein the TH expressing neurons are also immunoreactive for AADC.

62. The method of claim 43, wherein the TH expressing neurons do not express DBH.

63. The method of claim 43, wherein the neural progenitor cells are selected from the group consisting of:
(a) adult human central nervous system (CNS) cells;
(b) adult rodent CNS cells;
(c) human embryonic cells;
(d) human fetal cells;
(e) human embryonic or fetal forebrain cells; and
(f) embryonic stem cells.

64. A composition produced according to the method of claim 43, wherein the percentage of the cells in the produced cell population expressing tyrosine hydroxylase is greater than 5%.

65. A method for reseeding the composition according to claim 64 comprising trypsinization and seeding of the TH expressing cells.

66. A method for treating a mammal with a tyrosine hydroxylase-related deficiency, comprising administering the composition according to claim 64 into the CNS of the mammal.

67. A method for treating a mammal with a disease state of the central nervous system, comprising administering the composition according to claim 64 into the CNS of the mammal.

68. The method of claim 67, wherein the disease state of the central nervous system is Parkinson's disease.

69. The method of claim 66, wherein the administering is by transplantation.

70. The method of claim 67 wherein the administering is by transplantation.


SEQUENCE LISTING (6 sequences)

SEQ ID NO: 1 - DNA, 19 bp, Artificial
  TH primer 1
gcccccacctggagtactt

SEQ ID NO: 2 - DNA, 20 bp, Artificial
  TH primer 2
gcgtggcgtatacctccttc

SEQ ID NO: 3 - DNA, 20 bp, Artificial
  AADC primer 1
cggcattggcagataccact

SEQ ID NO: 4 - DNA, 20 bp, Artificial
  AADC primer 2
attccaccgtgcgagaacag

SEQ ID NO: 5 - DNA, 26 bp, Artificial
  DBH primer 1
cacgtactggtgctacattaaggagc

SEQ ID NO: 6 - DNA, 25 bp, Artificial
  DBH primer 2
aatggccatcactggcgtgtacacc